(19) United States
(12) Patent Application Publication
(10) Pub. No.: US 2016/0007878 A1
(43) Pub. Date: Jan. 14, 2016

(54) DEVICE AND METHOD FOR MONITORING PAIN

(71) Applicant: Washington University, St. Louis, MO (US)

(72) Inventors: Eric Leuthardt, St. Louis, MO (US); Neill Wright, St. Louis, MO (US); Dan Moran, St. Louis, MO (US); Guy Genin, St. Louis, MO (US); Ammar Hawasli, St. Louis, MO (US); Oliver Krengel, St. Louis, MO (US); Ulysses Chang, St. Louis, MO (US)

(21) Appl. No.: 14/798,031

(22) Filed: Jul. 13, 2015

Related U.S. Application Data
(60) Provisional application No. 62/023,825, filed on Jul. 12, 2014.

(51) Int. Cl. A61B 5/053; A61B 5/01; A61B 5/00; A61B 5/0205; A61B 5/024

(52) U.S. Cl. CPC A61B 5/0531; A61B 5/02055; A61B 5/024; A61B 5/6825; A61B 5/01; A61B 5/0022

(57) ABSTRACT

Devices and methods for monitoring pain in a subject, including a wearable device in communication with an analysis system that monitors pain in a subject using measurements of electrodermal activity in the subject are disclosed.

### CROSS REFERENCE TO RELATED APPLICATIONS

This application claims the priority of U.S. provisional application No. 62/023,825, filed Jul. 12, 2014, which is hereby incorporated by reference in its entirety.

### FIELD OF THE INVENTION

The disclosure is directed to devices and methods for monitoring pain in a subject. In particular, this disclosure is directed to a wearable device in communication with an analysis system that monitors pain in a subject via electrodermal activity in the subject.

### BACKGROUND OF THE INVENTION

Because pain is a perceived phenomenon, the objective monitoring of pain in a subject remains a challenging problem in the clinical setting. However, the perception of pain by a subject may be accompanied by sympathetic nervous system activity. Sympathetic nervous system activity may in turn be accompanied by changes in at least one or more physiological states of the subject which may be amenable to monitoring using one or more sensors.

One such physiological state is electrodermal activity, also known as galvanic skin response. Galvanic skin response, corresponding to a measured electrical resistance or impedance in response to an electrical current applied to the skin of the subject, is typically measured on an inner hand, on a bottom of a foot of the subject, or on the volar forearm region. These particular locations on the body are known to exhibit increased sweating under stress. The inner hand location is typically used to measure electrodermal activity because thermoregulatory sweating in this region is thought to occur only under extreme heat conditions, corresponding to ambient temperatures of over about 32 degrees Celsius.

Galvanic skin response is typically measured by applying an electrical potential difference between a pair of electrodes in contact with the skin of the subject and measuring the resulting current flowing through the skin between the two electrodes. The electrodes are typically attached to the distal fingertips of the index and middle fingers of the subject. It is thought that the fingertips are more amenable to attachment to the skin of the patient using known attachment means such as surgical tape or adhesive, and it is further thought that the fingertips may sweat more under stress than other measurement sites. Although less common, the electrodes may also be mounted to the palm of the subject.

In existing electrodermal activity measurement devices, direct current is typically used to measure skin conductance, defined herein as the inverse of electrical resistance. However, the measurement of skin conductance may be confounded by the build-up of an electric potential across capacitive elements within the skin. Skin potential may be used to assess electrodermal activity but these data have proven difficult to interpret. The measurement of alternating current through the skin induced by repeated cycles of applied voltage to assess impedance of the skin has been used in a limited number of devices. However, the complex data analysis accompanying this method has limited more widespread use.

The assessment of skin impedance to measure electrodermal activity may overcome at least some of the limitations accompanying other methods, such as the assessment of skin conductance under direct current conditions. In particular, the assessment of skin impedance may be better suited to assess a tonic component, the only component assessed via skin conductance methods, as well as a phasic component.

A need exists in the art for devices, systems, and methods for monitoring pain in a subject using an objective measure such as electrodermal activity.

### SUMMARY OF THE INVENTION

In an aspect, the invention encompasses a device to monitor pain in a subject. The device comprises: a portable unit comprising: a galvanic skin response sensor comprising: at least two electrodes held in a fixed position on the skin of the subject; at least one additional sensor; and an impedance converter operatively connected to the at least two electrodes; a microcontroller operatively connected to the impedance converter and to the at least one additional sensor wherein the microcontroller operates the impedance converter; and a wireless transmitter operatively connected to the microcontroller; and an analysis unit operatively connected to the portable unit, the analysis unit comprising: a wireless receiver; a computing device operatively connected to the wireless receiver, the computing device comprising: at least one processor; a computer readable media (CRM) encoded with a data analysis application executable on the at least one processor.

In another aspect, the invention encompasses a pain sensing system for estimating a pain index representative of a perceived pain in a subject. The system comprises: a portable unit comprising: a galvanic skin response sensor to monitor a skin impedance of the subject, the galvanic skin response sensor comprising; at least two electrodes maintained in a fixed position and separation distance on a portion of skin of the subject; at least one additional sensor; and an impedance converter to obtain a plurality of electrode measurements and analyze the plurality of electrode measurements to calculate at least one measured skin impedance value; a wireless data transmitter to transmit the plurality of electrode measurements, the at least one measured skin impedance, and a second plurality of additional sensor measurements to an analysis system; the analysis system comprising: a wireless data receiver to receive the plurality of electrode measurements, the at least one measured skin impedance, and a second plurality of additional sensor measurements; at least one processor; a CRM comprising a data analysis application comprising a plurality of modules executable on the at least one processor, wherein the data analysis application calculates a pain index using the plurality of electrode measurements, the at least one measured skin impedance, and the second plurality of additional sensor measurements.

In still another aspect, the invention encompasses A method to monitor pain in a subject. The method comprises: positioning a portable unit on a hand of a patient, the portable unit comprising: a galvanic skin response sensor comprising: at least two electrodes held in a fixed position on the skin of the subject; at least one additional sensor; and an impedance converter operatively connected to the at least two electrodes; a microcontroller operatively connected to the impedance converter and to the at least one additional sensor wherein the microcontroller operates the impedance converter; and a wireless transmitter operatively connected to the microcontroller; obtaining at least one electrode measurement from the palm of the subject; calculating at least one measured skin impedance for the subject using the at least one electrode measurement; receiving a plurality of signals from the microcontroller encoding raw data comprising: the at least one electrode measurement; the at least one measured skin impedance, at least one additional sensor measurement from the skin impedance of the subject, and any combination thereof; and transmitting a plurality of wireless signals encoding the raw data to the wireless receiver; analyzing raw data using an analysis unit operatively connected to the portable unit, the analysis unit comprising: a wireless receiver; a computing device operatively connected to the wireless receiver, the computing device comprising: at least one processor; a computer readable media (CRM) encoded with a data analysis application executable on the at least one processor.

### DESCRIPTION OF THE FIGURES

The application file contains at least one drawing executed in color. Copies of this patent application publication with color drawing(s) will be provided by the Office upon request and payment of the necessary fee.

The following figures illustrate various aspects of the disclosure.

FIG. 1 is a schematic diagram illustrating the signal flow among various elements of a pain monitor system.

FIG. 2 is a schematic diagram illustrating the various devices and elements of a pain monitor system.

FIG. 3A is an image illustrating a clip with associated electrodes.

FIG. 3B is an image illustrating a clip and associated electrodes attached to the palmar surface of a subject's hand over the subject's hypothenar eminence.

FIG. 4A is an image of a portable unit attached to the wrist and hand of a subject shown in a supinated view.

FIG. 4B is an image of a portable unit attached to the wrist and hand of a subject shown in a pronated view.

FIG. 4C is a schematic diagram illustrating various elements of the pain monitor system.

FIG. 5 is a graph summarizing changes in measured skin impedance during a pain stimulus.

FIG. 6 is a schematic diagram illustrating the elements of a pain monitor system.

While multiple embodiments are disclosed, still other embodiments of the present disclosure will become apparent to those skilled in the art from the following detailed description, which shows and describes illustrative embodiments of the disclosure. As will be realized, the disclosure is capable of modifications in various aspects, all without departing from the spirit and scope of the present disclosure. Accordingly, the drawings and detailed description are to be regarded as illustrative in nature and not restrictive.

### DETAILED DESCRIPTION

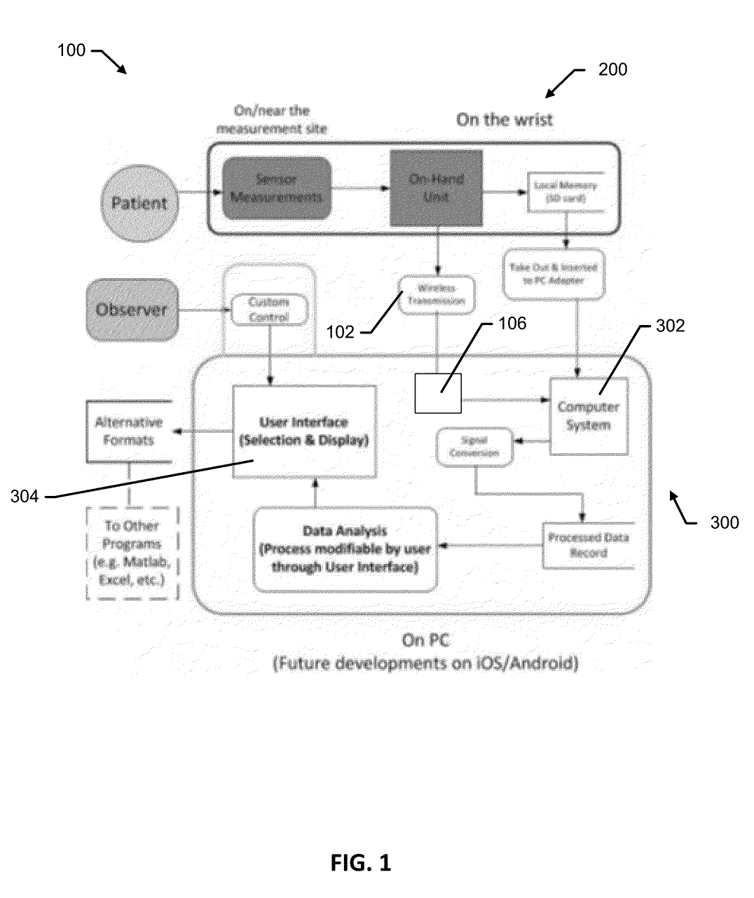

In various aspects, a pain monitor system 100 is provided to monitor pain in a subject. The pain monitor system 100 includes a portable unit 200 and an analysis unit 300 in communication with the portable unit 200, as shown in FIG. 1. The portable unit 200 may include a pain monitor device 400 that may include a galvanic skin response sensor 402 to measure the electrodermal activity and at least one additional sensor 404 to measure an additional physiological state and/or an activity of the subject. In a non-limiting example, the portable unit 200 may be wearable by the subject and may be in communication with the analysis unit 300 via a wireless data transmitter interface 102, thereby facilitating the freedom of movement of the subject. In additional aspects, a method of monitoring pain in a subject using the monitor device 400 is provided. The devices, systems, and methods are described in additional detail herein below.

### I. Portable Unit

The portable unit 200 may include the pain monitor device 400 in communication with the analysis unit 300 via the wireless data transmission interface 102. The portable unit 200 may further include a casing 202 containing at least a portion of the electrical components associated with the pain monitor device 400 and at least a portion of the wireless data transmission interface 102. The portable unit 200 may further include a fastening means including, but not limited to, a wrist band to fasten the casing 202 to the subject. In an aspect, the portable unit 200 may be worn on the wrist of the subject and the casing 202 may be substantially similar in size to the size of a wrist watch.

The portable unit 200 may transmit data to and from the analysis unit 300 by at least one means of data transmission. The analysis unit 300 may transmit and receive data via a wireless data transmission interface 102. In addition to the wireless data transmission interface 102, the portable unit 200 may also be connected to the analysis unit 300 by USB or RS232 serial connector to transfer data via a wired connection.

### A. Pain Monitor Device

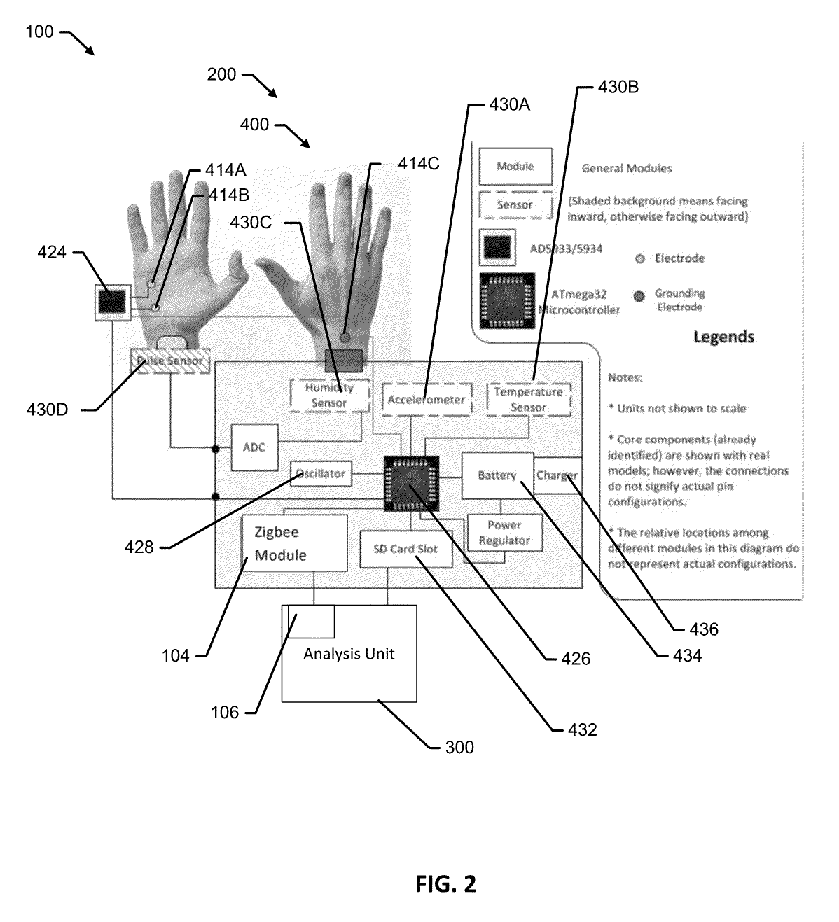

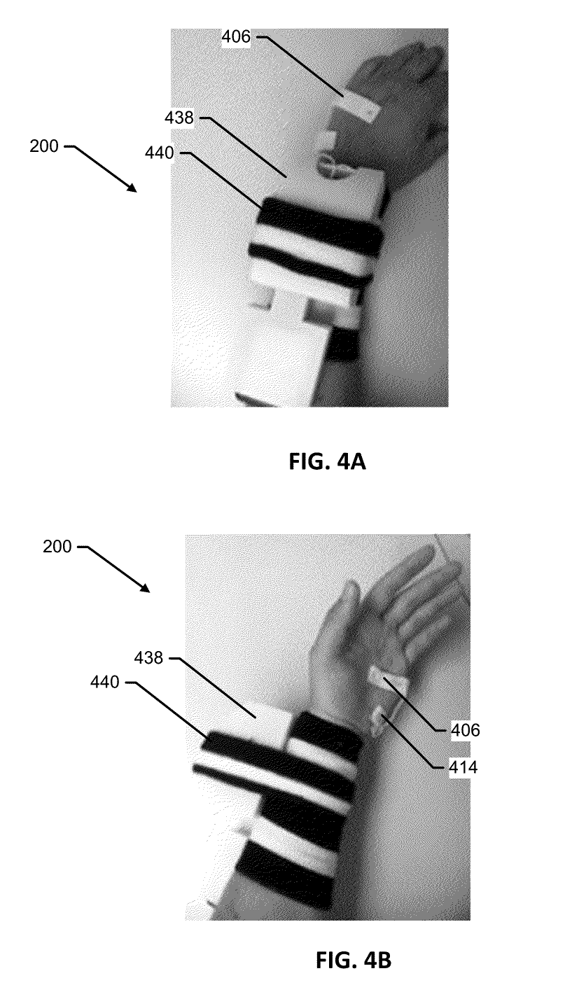

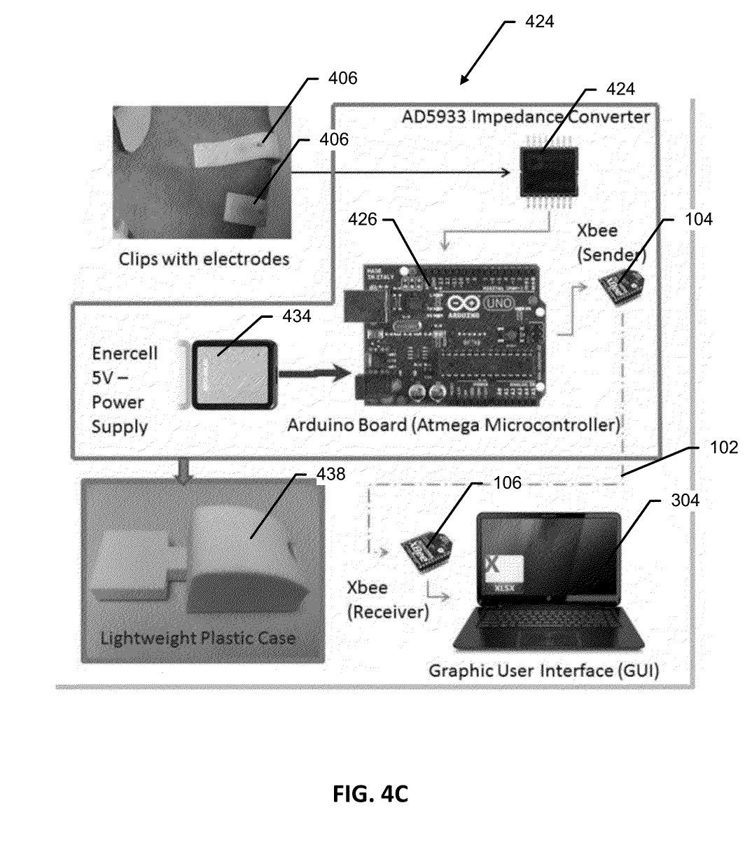

In various aspects, the portable unit 200 may include a pain monitor device 400. The pain monitor device 400 may include a galvanic skin response sensor 402 and at least one additional sensor 404 to measure at least one additional physiological state of a subject and/or the activity of a subject. The pain monitor device 400 may further include electrical components and associated circuitry to perform at least one or more additional functions including, but not limited to: operating the galvanic skin response sensor 402 and at least one additional sensor 404; communicating the measurements obtained from the sensors to the analysis unit 300 via the wireless data transmission interface 102 as illustrated in FIG. 2. At least a portion of the pain monitor device 400 may be contained within the casing 202 wearable by the user, as illustrated in FIGS. 4A and 4B. In one non-limiting example, the casing 202 containing various electrical components and associated circuitry may be worn around the wrist of the subject, as illustrated in FIGS. 4A and 4B.

i. Galvanic Skin Response Sensor (GSRU) (Electrodermal Activity Sensor)

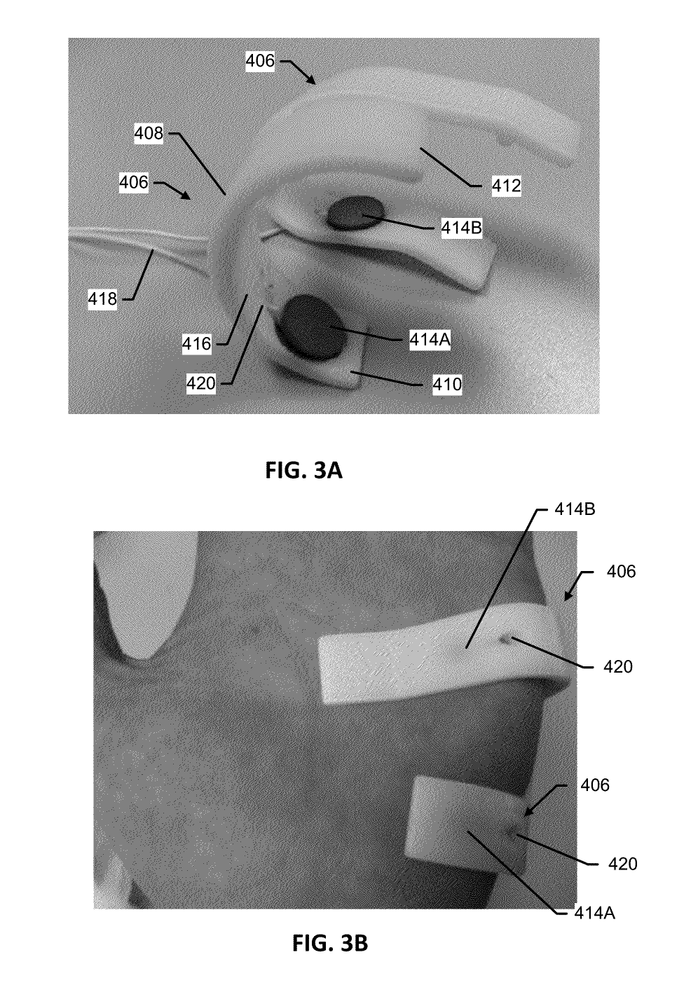

As illustrated in FIG. 2, the components of the galvanic skin response sensor 402 may include at least two electrodes 414A/414B affixed to a palm of the subject using one or more deformable clips 406, illustrated in FIG. 3A. The deformable clips 406 may hold the two or more electrodes 414A/414B of the galvanic skin response sensor 402 in fixed locations on the palm of the subject, as illustrated in FIG. 3B. Referring again to FIG. 3A, the galvanic skin response sensor 402 may further include a lead 418 operatively connected to the at least two electrodes 414A/414B. The lead 418 may transmit electrical signals between the electrodes 414A/414B and associated circuitry operatively connected to the lead 418 opposite to the electrodes 414A/414B. The associated circuitry of the galvanic skin response sensor 402 may perform various electrical measurements used to assess electrodermal activity in the subject, described in detail herein below. A more detailed description of the components of the galvanic skin response sensor 402 is provided herein below.

a) Electrodes

Referring again to FIG. 2, the galvanic skin response sensor 402 may include at least two electrodes 414A/414B affixed to a palm of the subject to conduct electrical signals to and from the subject via a lead 418 connected to associated circuitry in various aspects. The skin impedance of the subject may be assessed using electrical signals obtained from the at least two electrodes 414A/414B using any method known in the art without limitation. In one non-limiting example. a time-varying voltage cycle including, but not limited to an AC voltage may be applied to a first electrode 414A and the time-varying current across the skin of the subject between the first electrode 414A and a second electrode 414B may be measured. The time-varying voltage and time-varying current may be analyzed using any known method to determine the skin impedance of the subject.

The at least two electrodes 414A/414B may include a first electrode 414A and a second electrode 414B. The first electrode 414A may apply the time-varying voltage to the skin of the subject. The first electrode 414A and the second electrode 414B may also monitor the current across the skin of the subject between the first electrode 414A and the second electrode 414B induced by the applied time-varying voltage. In other aspects, the at least two electrodes 414A/414B may further include additional electrodes including, but not limited to a third electrode 414C.

The third electrode 414C may be attached to the skin of the subject in a separate region from the first electrode 414A and the second electrode 414B. The third electrode 414C may be used to provide additional measurements used to determine skin impedance. In various non-limiting examples, the third electrode 414C may function as a reference electrode, a grounding electrode, and/or a counter electrode.

In various aspects, the electrodes 414 may be provided in the form of any known electrode type suitable for non-invasively measuring current and voltage from the skin of a subject without limitation. In various aspects, the electrodes 414 may be provided in the form of disc electrodes ranging from about 1 mm to about 20 mm in diameter. In one aspect, the electrodes 414 may be non-polarizable electrodes including, but not limited to Ag—AgCl electrodes such as an In Vivo Metric (IVM) electrode. In an aspect, the first and second electrodes 414A/414B may be placed on the palmar surface of the subject at a separation distance ranging from about 0.25 inches to about 4 inches. In one aspect, the separation distance may be selected to cover the same dermatome area, which may depend on the size of subject's palm. In a non-limiting example, the electrodes may be placed within the hypothenar eminence area of the palmar surface of the subject. “Hypothenar eminence area”, as used herein, refers to the skin overlying the three hypothenar muscles of the palm that control the motion of the little finger; the hypothenar eminence area corresponds to the lateral edge of the hand extending between the 5^{th} metacarpal-little finger joint and the wrist joint.

In various aspects, the electrodes 414 may be placed in direct contact with the skin of the subject without the use of a conductive gel, adhesive, or any other intervening substance. In an aspect, the electrodes 414 may be held in a fixed position on the skin of the subject using a mechanical electrode fixation element including, but not limited to, at least one flexible clip 406.

b) Electrode Fixation Element

In various aspects, the electrodes 414 used obtain the measurements used to determine the skin impedance of a subject may be held in a fixed position on the skin of the subject using any known electrode fixation element without limitation. Without being limited to any particular theory, the fixed position of the electrodes 414 is thought to reduce motion artifacts in the skin impedance measurements and subsequent data analysis. Non-limiting examples of suitable electrode fixation elements include adhesives such as adhesive tapes and patches, and mechanical fixation elements such as wraps and flexible clips 406. In an aspect, the electrodes 414 are held in a fixed position on the skin of the subject using at least one flexible clip 406.

In an aspect, the galvanic skin response sensor 402 may include at least one flexible clip 406 to hold at least one electrode 414 in a fixed position on the palm of the subject. As illustrated in FIG. 3A, each clip 406 may include a strip 408 of a rigid, but elastic material ending in a first free end 410 and an opposed second free end 412. The strip 408 may be formed into an approximate “C” shape, with the first free end 410 and the second free end 412 aligned and separated at a separation distance from one another. The “C” shape of the clip 406 may be dimensioned to conform to a lateral edge of a subject's hand such that the clip 406 will slip over the edge of the subject's hand, as illustrated in FIG. 3B. In this aspect, the separation distance of the free ends 410/412 of the clip 406 may be dimensioned to a distance slightly less than the thickness of the subject's palm, such that the free ends 410/412 of the clip 406 may elastically deform away from one another as the clip 406 is placed over the edge of the subject's hand. This elastic deformation of the clip 406 induces downward elastic restoring forces at the free ends 410/412 that hold the clip 406 in place on the hand of the subject.

As illustrated in FIG. 3A, each clip 406 may include an electrode 414 attached to an inner surface 416 of the clip 406 near the first free end 410, such that the electrode 414 directly contacts the palm of the subject at a predetermined location when the clip 406 is slipped onto the hand of the subject. The electrode 414 may be electrically coupled to a lead 418 connecting the electrode 414 to the associated circuitry within the casing 202, which may be attached to the wrist of the subject during use. In an aspect, the strip 408 may be provided with an opening 420 through which the lead 418 may pass through the strip 408 to the associated circuitry in the casing 202.

In various aspects, the clip 406 may be formed from a strip 408 of any flexible material without limitation. In an aspect, the material of the clip 406 may be non-conductive to prevent interference with the electrical measurements performed by the electrode 414. In another aspect, the material of the clip 406 may be non-allergenic and biocompatible to prevent skin irritation or other dermatological reactions of the skin of the subject with the clip 406 during use. Non-limiting examples of suitable materials for the clip 406 include polymers such as polyethylene as well as metals. In an additional aspect, the clip 406 may be enclosed in a glove-like enclosure attached to a wrist band containing the casing 202 and associated electronics of the portable unit 200.

In an aspect, each clip 406 may be formed of a permanently deformable material to provide the ability to bend the clip 406 to adjust for variation in the hand size of the subjects. In another aspect, the clip 406 may be non-adjustable. In this other aspect, the clip 406 may be provided in a range of sizes to accommodate a range of hand sizes of the subject. In one aspect, the clip 406 may be provided in at least three sizes to fit a majority of palm sizes of subjects.

In one aspect, each clip 406 may hold one electrode 414 as illustrated in FIG. 3A. In this one aspect, the galvanic skin response sensor 402 may include at least two clips 406 and a single electrode 414 may be attached to each clip 406. In another aspect, a single clip 406 may hold a first electrode 414A and a second electrode 414B. By way of non-limiting example, the clip 406 may be provided in the form of a sheet formed into an approximate half-cylinder, in which the electrodes 414A/414B are attached to the inner surface of the sheet near one edge and the palm of the subject is inserted between the edges of the sheet into the interior of the half cylinder. In another non-limiting aspect, two strips 408 formed into individuals “C” shapes may be joined by one or more cross-members to maintain the strips 408 at a fixed distance and orientation relative to one another.

In an aspect, the clip 406 may further include an additional electrode 414C (not shown) attached to the inner surface 416 near the second free end 412 opposite to the first free end 410. In this aspect, the additional electrode 414C may be placed in contact with the back of the hand opposite to the pair of electrodes 414A/414B used to measure electrodermal activity. This additional electrode 414C may function as a reference electrode, grounding electrode, and/or a counter electrode to reduce noise in the measurements of the first electrode 414A and second electrode 414B.

c) Associated Circuitry

The galvanic skin response sensor 402 may further include associated circuitry operatively attached to the at least two electrodes 414 via the lead 418 to conduct the electrical measurements used to assess the skin impedance of the subject. Any known circuitry suitable for conducting skin galvanic response measurements may be used in the galvanic skin response sensor 402 without limitation. In one aspect, the associated circuitry may include a dedicated element including, but not limited to, an impedance converter 422. In another aspect, the associated circuitry may be incorporated into a device including, but not limited to, a microprocessor 426 to conduct the electrical measurements used to assess the skin impedance of the subject as well as to perform additional functions described herein below. The associated circuitry may be operatively coupled to the at least two electrodes 414 via the lead 418. The lead 418 may include a single cable or other conductive element with branches connected to each of the at least two electrodes 414 in one aspect. In another aspect, the lead 418 may include at least two separate cables, each cable connected to one of the at least two electrodes 414.

In various aspects, the measurements performed by the associated circuitry may include, but are not limited to: applying voltage in repeating cycles to a first electrode 414A and monitoring an oscillating current through the sin of the subject between the first electrode 414A and the second electrode 414B induced by the applied oscillating voltage. In various other aspects, the additional circuitry may analyze the electrical measurements over time to determine the skin impedance of the subject.

### Impedance Converter

Referring to FIG. 2, the associated circuitry 422 of the galvanic skin response sensor 402 may include an impedance converter 424 operatively coupled to the microcontroller 426. The impedance controller 424 may perform any one or more of the following functions including, but not limited to: receive control signals from the microcontroller 426; generate a series of voltage cycles; deliver the series of voltage cycles to a first electrode 414A; monitor the current cycles between the first electrode 414A and the second electrode 414B induced by the applied voltage cycles; analyze the series of voltage cycles and current cycles to determine a series of impedances; and transmit the series of impedances to the microcontroller 426. Any known impedance converter device may be incorporated into the associated circuitry 422 of the galvanic skin response sensor 402 without limitation including, but not limited to an AD5933/5934 impedance converter (Analog Devices, Inc., Norwood, Mass., USA).

In various aspects, each voltage cycle may include an alternating voltage cycle characterized by: a alternating voltage ranging from about 0.5 V peak-to-peak to about 5 V peak-to-peak and a frequency ranging from about 500 Hz to about 4000 Hz. In one aspect, the voltage cycle may include an alternating voltage cycle characterized by an alternating voltage of about 1.95 V peak-to-peak and a frequency of about 1350 Hz. In various aspects, the voltage cycles may be applied to the at least one electrode 414 for between about 100 settling cycles and about 1000 settling cycles. In one aspect, the voltage cycles may be applied to the at least one electrode 414 for about 500 settling cycles. The waveform of the voltage cycles may be specified using any known method without limitation including, but not limited to, the use of an oscillator 428 operatively connected to the impedance converter 424 via the microcontroller 426, as illustrated in FIG. 2.

In various aspects, the impedance converter 424 may analyze the applied voltage and induced current measurements over all settling cycles to assess the skin impedance of the subject using any known impedance analysis method without limitation. In one aspect, the applied voltage and induced current measurements may be analyzed by performing a frequency-domain analysis of the measurements. In one aspect, the voltage cycles and the current cycles may be analyzed by performing a discrete Fourier transformation (DFT) of the series of voltage cycles and the series of current cycles. In this aspect, the results of the DFT analysis may yield two raw data values corresponding to the real (R) and imaginary (I) portions of the DFT function generated for each voltage cycle and associated current cycle. In this aspect, the two raw data values may be used to calculate the measured magnitude and phase of the impedance using any known method.

By way of non-limiting example, the magnitude of the DFT function may be calculated using the real and imaginary portions of the DFT function according to Eqn. (1):

DFT Magnitude=\sqrt{R²+I²} Eqn. (1)

The magnitude of the measured skin impedance may be calculated using Eqn. (2):

$$Skin Impedance = \frac{1}{Gain factor X DFT Magnitude} \qquad Eqn . ( 2 )$$

The inclusion of the gain factor in Eqn. (2) compensates for electrical impedance associated with the impedance converter 424 that may introduce an artifact into the skin impedance magnitude. The gain factor may be determined by performing calibration measurements in which a resistor of known resistance bridges the electrodes to obtain DFT magnitude and impedance measurements, which may be substituted into Eqn. (3):

$$Gain Factor = \frac{⌈ \frac{1}{Impedance} ⌉}{DFT Magnitude} \qquad Eqn . ( 3 )$$

The total measured phase (φtotal) of the impedance may be calculated using Eqn. (4):

φtotal(rad)=tan⁻¹(I/R) Eqn. (4)

The total measured phase (φtotal) calculated in Eqn. (4) may include the phase shift introduced by the impedance converter 424 (φsystem) as well as the phase of the impedance of the skin (φskin) of the subject. Calibration measurements similar to those used to obtain the gain factor described herein above may be used to determine φsystem. The phase introduced by the impedance converter (φsystem) may be subtracted from the total measured phase (φtotal) to determine the phase of the skin impedance phase (φskin) according to Eqn. (5):

φskin=φtotal−φsystem Eqn. (5)

In an additional aspect, the series of voltage cycles and current cycles measured by the electrodes 414 may be transmitted to the analysis unit 300. In this aspect, the analysis unit 300 may perform a similar frequency analysis to the method performed by the impedance converter 424 described herein previously to determine a magnitude and/or phase of the skin impedance of the subject.

### Microcontroller

In various aspects, a microcontroller 426 may coordinate the operation of the galvanic skin response sensor 402 and additional sensors 404, including the delivery of the sensor outputs to a storage medium 432 and/or wireless data transmitter 104 in communication with the analysis unit 300. In one aspect, the microcontroller 426 may operate the galvanic skin response sensor 402. In this one aspect, the microcontroller 426 may operate an impedance converter 424, receive impedance data measured from the electrodes 414 and converted by the impedance converter 424, receive impedance data measured from the electrodes 414 and converted by the impedance converter 424, and store the impedance data and/or deliver the impedance data to a transmitter 104 in communication with the analysis unit 300.

In another aspect, the microcontroller 426 may operate the galvanic skin response sensor 402 by generating a series of voltage cycles, delivering the series of voltage cycles to a first electrode 414A, receiving a series of current cycles measured between the first electrode 414A and the second electrode 414B induced by the series of applied voltage cycles, and delivering the series of voltage cycles and the measured series of current cycles to a storage medium 432 and/or a transmitter 104 in communication with the analysis unit 300. Referring to FIG. 2, the microcontroller 426 may be operatively connected to an oscillator 428 that generates a predetermined waveform that defines the voltage cycle delivered to the electrodes 414.

Any suitable microcontroller 426 known in the art may be incorporated for use in the galvanic skin response sensor 402 without limitation. In one aspect, a commercially available microcontroller including, but not limited to: an ATmega microcontroller may be incorporated for use in the galvanic skin response sensor 402.

ii. Additional Sensors

In various aspects, the pain monitor device 400 may further include at least one additional sensor 430 to monitor movement, external environment, and/or at least one additional physiological state of the subject. Non-limiting examples of the at least one additional sensor 430 includes: a motion sensor or accelerometer, a humidity sensor, a temperature sensor, a pulse sensor, and any other suitable physiological sensor. In one aspect, measurements obtained from the at least one additional sensor 430 may be used to reduce artifacts associated with the impedance measurements. In another aspect, the at least one additional sensor 430 may provide supplemental information regarding the sympathetic nervous system activity of the subject. In a non-limiting example, at least one additional sensor 430 may be incorporated or attached to the flexible clip 406 or other electrode fixation element.

### Motion Sensor

In an aspect, the at least one additional sensor 430 may include a motion sensor 430A including, but not limited to, an activity sensor or accelerometer. Data measured by the motion sensor 400A may include, but is not limited to: linear and/or rotational accelerations in any direction without limitation. The data measured by the motion sensor 430A may be used to reduce variability in impedance measurements due to swinging of the subject's hand, which may vary the degree of contact of the electrodes 414 with the skin of the subject. The motion sensor 430A may be operatively coupled to the microprocessor 426; the microprocessor 426 may operate the motion sensor 430A, receive measurements from the motion sensor 430A, and store and/or transmits measurements received from the motion sensor 430A to the analysis unit 300 as illustrated in FIG. 2.

### Temperature Sensor

In an aspect, the at least one additional sensor 430 may include a temperature sensor 430B. Data measured by the temperature sensor 430B may include, but is not limited to: ambient temperature and/or skin temperature of the subject. The data measured by the temperature sensor 430B may be used to assess any potentially confounding effects of thermoregulatory perspiration in the region of the electrodes 414. In addition, the data measured by the temperature sensor 430B may be used to determine the degree of thermoregulatory activity and/or other physiological manifestations of sympathetic nervous system activity related to thermoregulation. The temperature sensor 430B may be operatively coupled to the microprocessor 426; the microprocessor 426 may operate the temperature sensor 430B, receive measurements from the temperature sensor 430B, and store and/or transmits measurements received from the temperature sensor 430B to the analysis unit 300 as illustrated in FIG. 2.

### Humidity Sensor

In an aspect, the at least one additional sensor 430 may include a humidity sensor 430C. Data measured by the humidity sensor 430C may include, but is not limited to, ambient absolute humidity and/or relative humidity in the vicinity of the subject. The data measured by the humidity sensor 430C may be used to assess any potentially confounding effects of thermoregulatory perspiration in the region of the electrodes 414. The humidity sensor 430C may be operatively coupled to the microprocessor 426; the microprocessor 426 may operate the humidity sensor 430C, receive measurements from the humidity sensor 430C, and store and/or transmit measurements received from the humidity sensor 430C to the analysis unit 300 as illustrated in FIG. 2.

### Pulse Sensor

In an aspect, the at least one additional sensor 430 may include a pulse sensor 430D. Data measured by the pulse sensor 430D may include, but is not limited to, a pulse rate of the subject. The data measured by the pulse sensor 430D may be used to assess any potentially confounding effects of pulse rate and/or other physiological manifestations of sympathetic nervous system activity on the measured skin impedance of the subject. The pulse sensor 430D may be operatively coupled to the microprocessor 426; the microprocessor 426 may operate the pulse sensor 430D, receive measurements from the pulse sensor 430D, and store and/or transmit measurements received from the pulse sensor 430D to the analysis unit 300 as illustrated in FIG. 2.

In one aspect, the additional sensors 430 may be provided in the form of separate instruments. In another aspect, one or more of the additional sensors 430 may be combined such that one instrument may monitor two or more of the impedance, activity, ambient conditions, and/or physiological states of the subject. By way of non-limiting example, the galvanic skin response sensor 402 may be operated using an impedance converter 424 that further includes a temperature sensor used to measure ambient temperature and/or skin temperature of the subject.

### B. Additional Electrical Components

Referring again to FIG. 2, the portable unit 200 may further include one or more additional electrical components including, but not limited to: a wireless transmitter 104 to communicate data between the portable unit 200 and the analysis unit 300; local memory 432 to store data measurements for later analysis and/or transfer to other devices; and a power source 434 to provide electrical power to the various elements, sensors, and devices of the pain monitor device 400. In an aspect, the portable unit 200 may include a printed circuit board (not shown) to provide any one or more of the functions of the various elements, sensors, and devices of the pain monitor device 400 described herein. In this aspect, the printed circuit board may provide the functionality of the portable unit 200 in a relatively compact space.

i. Wireless Transmitter

In various aspects, a wireless transmitter 104 may be operatively coupled to wirelessly transmit data to a corresponding wireless receiver 106 in the analysis unit 300. The transmitter 104 provides for communication of signals encoding data between the microcontroller 426 of the portable unit 200 and the computing devices of the analysis unit 300. Any wireless communication network architectures may be used to communicate data via the wireless transmitter 104 without limitation including, but not limited to point to point (PTP) wireless communication networks and multipoint wireless communication networks. In one aspect, the wireless transmitter 104 may support a basic IEEE 802.15.4 protocol with further upper layers to provide secure data transmission. In another aspect, the wireless transmitter 104 may support a Zigbee secure protocol, which provides layers of security and secure transmission of data.

In an aspect, the size of the transmitter 104 may range from about 0.5 cm to about 2 cm. In another aspect, the transmitter 104 may be sufficiently small to fit within a wrist band of the portable unit 200. The transmitter 104 may provide high-powered or low-powered signal transmission, depending on the particular needs of the pain monitoring system 100. The transmitter 104 may be able to transmit over long distances via long-range transmission, or by passing data through intermediate devices to reach more distant devices operatively connected in a mesh network. The data transmission range may vary from about 100 feet up to about 28 miles. In one aspect, an extender may be used to extend the range of the transmitter 104 up to 40 miles. Depending on the data transmission range, the power consumption of the transmitter 104 may range from about 1 μA to about 24 μA. In a non-limiting example, the wireless transmitter 104 may be an Xbee transmitter which transmits to a corresponding Xbee receiver within the analysis unit 300 by way of a Zigbee secure module.

ii. Local Memory

In an aspect, the portable unit 200 may further include local memory 432 operatively coupled to the microprocessor 426 as illustrated in FIG. 2. Non-limiting examples of suitable local memory 432 elements include: internal memory storage and/or removable memory devices. The local memory 432 may include any suitable computer readable medium without limitation. In one aspect, the local memory 432 may be provided in the form of an SD card. In this aspect, the portable unit 200 may further include an SD card slot operatively coupled to microcontroller 426, as illustrated in FIG. 2. In a non-limiting example, a local memory 432 of at least 1 GB may be incorporated into the portable unit 200.

### Power Source

In an aspect, the portable unit 200 may further include a power source 434 to provide power to the electrical elements and devices of the portable unit 200. Any known suitable electrical power source may be included in the portable unit 200 including, but not limited to: rechargeable batteries, non-rechargeable batteries, and/or solar cells. In one aspect, the power source 434 may be a rechargeable battery. In this aspect, the portable unit 200 may further include a battery charger element 436 operatively coupled to the battery 434, as illustrated in FIG. 2. In one non-limiting example, the power source 434 may be a 5V rechargeable battery.

### C. Casing and Fastening Means

Referring to FIGS. 4A and 4B, the portable unit 200 may include a casing 202 containing at least a portion of the electrical components described herein above, and a fastening means 204 to fasten the casing 202 to the subject. In various aspects, the fastening means 204 may fasten the casing 202 in proximity to the clips 406 and electrodes 414 attached to the palm of the subject, thereby maintaining the casing 202 in an essentially fixed position relative to the clips 406 and electrodes 414. In one aspect, the fastening means 204 may be a wrist band attached to the casing 202. In this aspect, the wrist band may reversibly fasten the casing 202 to the wrist of the subject in proximity to the clips 406 and electrodes 414 attached to the hand of the subject.

i. Casing

The casing 202 may be any known casing 202 sized to contain at least a portion of the electrical components of the portable unit 200. In one aspect, the casing 202 may be comparable in size to a wristwatch. In an aspect, the material of the casing 202 may be non-conductive to prevent interference with operation of the electrical components of the portable unit 200. In another aspect, the material of the casing 202 may be non-allergenic and biocompatible to prevent skin irritation or other dermatological reactions of the skin of the subject with the clip 406 during use. In yet another aspect, the casing 202 may be constructed to be water-resistant using any known materials and methods without limitation. In this aspect, the casing 202 may be constructed from a water-proof material and may further incorporate gaskets, seals, and/or any other known water-resistant features.

ii. Fastening Means

The fastening means 204 may include any suitable fastening means known in the art without limitation including, but not limited to: a strap and interlocking buckle, VELCRO tape, reversibly adhesive tape, an elastic wrap, interlocking snaps, and any other suitable fastening means. In a non-limiting example, the portable unit 200 may include a glove-like fastening means 204 that may be worn over the hand of a subject. In one aspect, the fastening means 204 may be a wrist band, as illustrated in FIGS. 4A and 4B.

In various aspects, a wrist band may be used to attach the portable unit 200 to the arm of the subject and may further hold the casing 202 and clips 406 in a fixed spatial relationship. In this aspect, the wrist band may include additional features including, but not limited to an amount of padding to provide comfort during long wearing periods.

### II. Analysis Unit

Referring to FIG. 1, the pain monitor system 100 further includes an analysis unit 300 in communication with the portable unit 200 via the wireless data transmission interface. In an aspect, the analysis unit 300 may include a wireless receiver 106 operatively connected to a computing device 302 to receive data from the wireless transmitter 104 of the portable unit 200. The analysis unit 300 may further include a user interface unit 304 operatively connected to the computing device 302 that may include one or more user interface devices including, but not limited to: monitors, keyboards, mice, touchscreens, and any other known user interface device.

The wireless receiver 106 may be any known wireless receiving device without limitation. In various aspects, the wireless receiver 106 may be selected to be compatible with the transmission range, rate of data transmission, data transmission network devices and architecture, data transmission protocols, and any other relevant aspect of the wireless data transmitter 104 as described previously herein. In a non-limiting example the receiver 106 may be a Zigbee receiver employing a Zigbee protocol.

The computing device 302 of the analysis unit 300 may include a computer readable memory (CRM) encoding a data analysis application that includes one or more modules to: process the measured skin impedance data received from the portable unit 200; display and manipulate the skin impedance data received from the portable unit 200; and store the data skin impedance data received from the portable unit 200 and/or generated by the one or more modules of the data analysis application. The modules of the data analysis application may execute on one or more processors of the computing device 302.

In various aspects, the modules of the data analysis application may include: a signal processing module to convert signals received from the portable unit 200 into useable form; a data quality module to eliminate at least one artifact from the series of skin impedances using a correction rule and the at least one additional series of at least one additional sensor measurement of a physiological state of the subject or ambient environmental states to produce a series of processed skin impedances; a calibration module to convert the series of processed skin impedances into a series of pain indices representing the perceived pain of the subject according to a calibration rule; and a GUI module to generate one or more forms used to receive inputs to the analysis unit 300 and to deliver output to the user interface 304 of the data analysis unit 300.

In an aspect, the data quality module of the data analysis module may eliminate at least one artifact from the series of skin impedances using a correction rule that may be estimated using any applicable known relationships. By way of non-limiting example, measurements of pulse rate may indicate sympathetic nervous system activity unrelated to the perception of pain. In this example, changes in skin impedance accompanied by changes in pulse rate may be indicative of an artifact introduced by arousal or other phenomena unrelated to the perception of pain. As a result, a correction rule may assign a weight to changes in measured skin impedance accompanied by accelerated pulse rates that is lower than changes in measured skin impedance accompanied by no changes in measured pulse rate. In other aspects, the correction rules used to eliminate the at least one artifact may be empirically derived based on controlled experiments measuring the changes in skin impedance associated with various combinations of pain, temperature, humidity, movement, pulse rate, and any other relevant factor.

The calibration rule used to convert the processed skin impedances generated by the data quality module into pain index values may be estimated using any known applicable relationship or may be empirically derived as described herein previously. In various aspects, the pain index may be provided as a relationship between the skin impedance and the perceived pain of the subject. Any measurement of skin impedance may be incorporated into the calibration rule in any combination, including, but not limited to: impedance magnitude, rate of change of impedance magnitude, changes in impedance magnitude, changes in impedance magnitude relative to a baseline magnitude, impedance phase, rate of change of impedance phase, changes in impedance phase, changes in impedance phase relative to a baseline magnitude, and any combination threreof.

The output of the GUI module may displayed in any format by the user interface 304 without limitation including, but not limited to graphs, tables, and any other known data format. In various aspects, the output displayed by the user interface 304 may provide a pain index of a subject at one instant in time, a series of pain indices of a subject over a period of time, a comparison of pain indices of two or more subjects at an instant or over a period of time, and any other relevant output.

### III. Method of Monitoring Pain

In various aspects, a method of monitoring pain in a subject using the devices and systems described herein above are provided. The method may include: choosing the subject to be monitored; affixing the portable unit 200 to the subject; measuring the skin impedance, physiological parameters, and/or ambient environmental factors of the subject using the portable unit 200; and analyzing the data obtained by the portable unit 200 over a period of time. In an aspect, the analysis of the data may be performed using a data analysis application of a data analysis unit 104 operatively connected to the portable unit 200 via a wireless data transmission interface 102.

### A. Affix Portable Device to Subject

The portable unit 200 may be affixed to the arm of the subject using a fastening means 204 such as a wrist band. The clips 406 with electrodes 414 of the galvanic skin response sensor 402 may be slipped over the palm of the subject as shown in FIG. 3. The casing 202 with other components of the portable unit 200 may be slipped on with the wrist band. The clips 406 may be adjusted in place to ensure that the electrodes 414 are maintained in a fixed position on the palm of the subject. The electrodes 414 may be placed in the palmar region of the hand, including, but not limited to the hypothenar eminence. The electrodes 414 may be adjusted to be separated by a distance ranging from about 0.5 inches to about 3 inches based on the palm size of the subject, and the amount of current desired within the skin of the patient.

B. Obtain Measurements from Sensors

Once the portable unit 200 is in place, the portable unit 200 may be activated to conduct skin impedance measurements as described herein above. Other physiological sensors 430 including, but not limited to temperature sensors 430B, humidity sensors 430C and motion sensors 430A may also be activated. The subject may be monitored for a predetermined period and baseline. In an aspect, baseline resting data may be collected and analyzed to provide information used to calibrate the system 100. In one aspect, in the absence of subject-specific baseline data, pre-existing data from resting normal subjects may be used to calibrate the system 100.

The portable unit 200 may be operatively connected to a timer so that the skin impedance measurements are collected over a predetermined period of time in one aspect. In another aspect, the collection of skin impedance data may be manually started and stopped at a user-specified time.

### C. Transmit Skin Impedance Data to Analysis Unit

In an aspect, the sensor measurements collected by the portable unit 200 and/or the skin impedance measurements generated by the impedance converter 424 may be transmitted to the data analysis unit 300 for additional analysis, display, and/or storage. The data may be transmitted in real time to the analysis unit 300 via the wireless data transmission interface 102 or may be transmitted at predetermined intervals ranging from 1 to 30 minutes. The skin impedance data may also be stored in the local memory 432 of the portable unit 200, for a period ranging from about one day to about one month.

### D. Analyze Skin Impedance Data

In an aspect, the data received at the analysis unit 300 may be analyzed using the one or more modules of the data analysis application resident within the computing device 302 of the data analysis unit 300. As described previously herein, the modules of the data analysis application may calculate a pain index representing the perceived pain of the subject using the methods described herein above. In an aspect, the modules of the data analysis application may process the magnitude and/or phase of the skin impedance measured by the galvanic skin response sensor 402 and may further process measurements from the one or more additional sensors 430 to eliminate artifacts due to physiological and environmental factors including, but not limited to humidity, temperature, pulse and/or movement. In an aspect, the measured skin impedance data and measurements obtained from the at least one additional sensor 430 may be normalized relative to corresponding baseline or resting measurements.

### E. Monitor Multiple Subjects

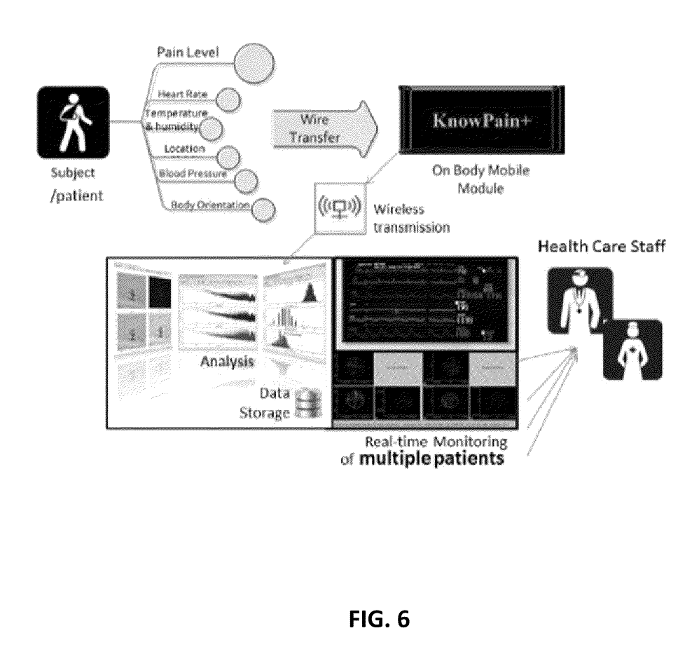

Referring to FIG. 6, skin impedance measurements from multiple subjects may be monitored using the systems and methods described herein above. In one aspect, multiple subjects may each be fitted with an affixed portable unit 200 to monitor the pain perceived by each subject. In one aspect, the skin impedance measurements from the multiple portable units 200 may be transmitted via the wireless data transmission interface 102 and may be received by a single data analysis unit 300. In another aspect, the skin impedance measurements from the multiple portable units 200 may be transmitted via the wireless data transmission interface 102 to multiple data analysis units 300 arranged in a network architecture. In this other aspect, each data analysis unit 300 may transmit data to any one or more of the other data analysis unit 300 in the network using known data transmission means including, but not limited to Internet data transfer. By way of non-limiting example, multiple portable units 200 units may be monitored simultaneously in a hospital setting, and the user interface 304 of the data analysis unit 300 may issue an alarm or other suitable indication to a medical practitioner if a pain index exceeds a predetermined threshold value, indicating a need for treatment.

The pain index data may be monitored and collected over a period of time, both as raw data from the galvanic skin response sensor 402, as well as the data generated by the data analysis unit 300 including, but not limited to pain index data. By way of non-limiting example, the data may be monitored over the full hospital stay of a subject if used at a hospital. The portable unit 200 may also store data within the local memory 434 as described herein above. By way of non-limiting example, the portable unit 200 may store the data locally when the subject is out of the wireless range of the analysis unit 300. The stored data may be periodically transferred to the application on the analysis unit 300.

### EXAMPLE

A pain monitor system similar to the system 100 described herein above was used to monitor the skin impedance of a subject during a pain stimulus. A portable unit 200 was affixed to the arm of a subject as described herein above and illustrated in FIGS. 4A and 4B. After activating the portable unit 200, a pain stimulus was applied to the free hand of the subject in the form of submersion in ice water for a period of about 5 minutes. The skin impedance of the subject was monitored during the pain stimulus, as well as for a period of about 16 minutes after removal of the pain stimulus.

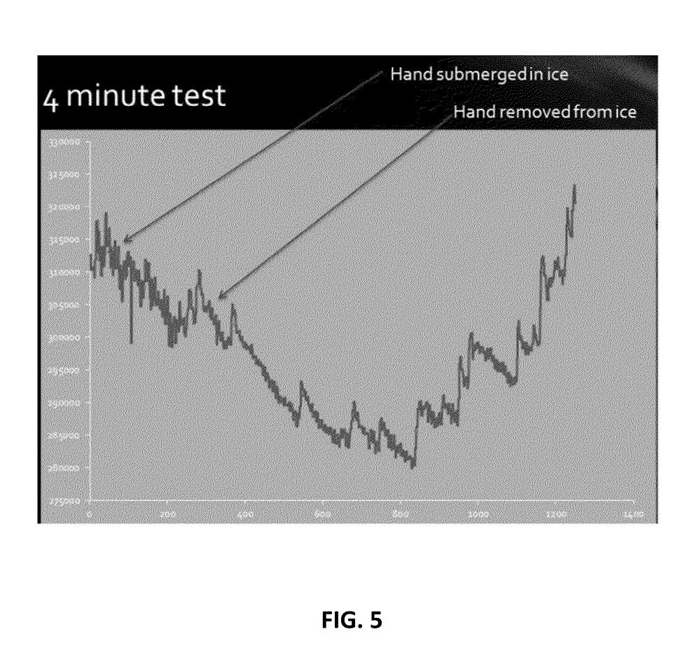

FIG. 5 is a summary of the skin impedance of the subject measured by the portable unit before, during, and after the pain stimulus. The skin impedance decreased during the initial exposure of the subject to the pain stimulus and continued to decrease for about 8 minutes after removal of the pain stimulus. The measured skin impedance rebounded to the baseline value during the period between 500 seconds and 900 seconds after removal of the pain stimulus.

The results of this experiment demonstrated that skin impedance of a subject decreased in response to exposure to a pain stimulus.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

We claim the following:

1. A device to monitor pain in a subject, the device comprising:
a portable unit comprising:
a galvanic skin response sensor comprising:
at least two electrodes held in a fixed position on the skin of the subject;
at least one additional sensor; and
an impedance converter operatively connected to the at least two electrodes;
a microcontroller operatively connected to the impedance converter and to the at least one additional sensor wherein the microcontroller operates the impedance converter; and
a wireless transmitter operatively connected to the microcontroller; and
an analysis unit operatively connected to the portable unit, the analysis unit comprising:
a wireless receiver;
a computing device operatively connected to the wireless receiver, the computing device comprising:
at least one processor;
a computer readable media (CRM) encoded with a data analysis application executable on the at least one processor.

2. The device of claim 1, further comprising at least one clip to hold the at least two electrodes held in a fixed position on the skin of the subject, wherein each clip of the at least one clips comprises a strip of a flexible material formed into a C-shape.

3. The device of claim 1, wherein each electrode of the at least two electrodes is attached to an inner surface of the at least one clip.

4. The device of claim 2, wherein the at least one clip fits over a hypothenar eminence of a hand of the subject.

5. The device of claim 1, wherein the at least one additional sensor is selected from the group consisting of a temperature sensor, a humidity sensor, a pulse rate sensor, a motion sensor, and any combination thereof.

6. The device of claim 1, wherein the at least one additional sensor monitors at least one additional state selected from the group consisting of a physiological state of the subject, a motion of the subject, and an ambient environmental condition.

7. The device of claim 1, wherein the impedance converter obtains at least one electrode measurement from the skin of the subject and calculates at least one measured skin impedance for the subject using the at least one electrode measurement.

8. The device of claim 7, wherein the wireless transmitter:
receives a plurality of signals from the microcontroller encoding raw data comprising: the at least one electrode measurement; the at least one measured skin impedance, at least one additional sensor measurement from the skin impedance of the subject, and any combination thereof; and
transmits a plurality of wireless signals encoding the raw data to the wireless receiver.

9. The device of claim 8, further comprising a local storage module operatively connected to the microcontroller to store at least a portion of the raw data.

10. The device of claim 8, wherein the wireless receiver receives the plurality of wireless signals encoding the raw data from the wireless transmitter.

11. The device of claim 8, wherein the data analysis application calculates a pain index using the raw data.

12. The device of claim 8, wherein the data analysis application comprises a plurality of modules executable on the one or more processors, the plurality of modules comprising:
a signal processing module to convert the series of raw impedances into a series of input impedances;
a data quality module to eliminate at least one artifact from the series of input impedances using a correction rule and the at least one additional series of at least one additional raw physiological state of the subject to produce a series of processed impedances;
a calibration module to convert the series of processed impedances into a series of pain indices; and
a GUI module to generate one or more forms used to receive inputs to the analysis system and to deliver output from the analysis system.

13. The device of claim 1, wherein the wireless receiver operates on a Zigbee protocol.

14. The device of claim 1, wherein the microcontroller is an ATmega microcontroller.

15. A pain sensing system for estimating a pain index representative of a perceived pain in a subject, the system comprising:
a portable unit comprising:
a galvanic skin response sensor to monitor a skin impedance of the subject, the galvanic skin response sensor comprising;
at least two electrodes maintained in a fixed position and separation distance on a portion of skin of the subject;
at least one additional sensor; and
an impedance converter to obtain a plurality of electrode measurements and analyze the plurality of electrode measurements to calculate at least one measured skin impedance value;
a wireless data transmitter to transmit the plurality of electrode measurements, the at least one measured skin impedance, and a second plurality of additional sensor measurements to an analysis system;
the analysis system comprising:
a wireless data receiver to receive the plurality of electrode measurements, the at least one measured skin impedance, and a second plurality of additional sensor measurements;
at least one processor;
a CRM comprising a data analysis application comprising a plurality of modules executable on the at least one processor, wherein the data analysis application calculates a pain index using the plurality of electrode measurements, the at least one measured skin impedance, and the second plurality of additional sensor measurements.

16. The system of claim 15, wherein the at least one additional sensor monitors at least one additional state selected from the group consisting of a physiological state of the subject, a motion of the subject, and an ambient environmental condition.

17. The system of claim 15, wherein the plurality of modules comprise:
a signal processing module to smooth and/or filter the plurality of electrode measurements, the at least one measured skin impedance, and a second plurality of additional sensor measurements;
a data quality module to eliminate at least one artifact from the at least one measured skin impedance using a correction rule and second plurality of additional sensor measurements to produce at least one processed impedances;
a calibration module to convert the at least one processed impedances into at least one pain index value; and
a GUI module to generate one or more forms used to receive inputs to the analysis system and to deliver output from the analysis system.

18. A method to monitor pain in a subject, the method comprising:
positioning a portable unit on a hand of a patient, the portable unit comprising:
a galvanic skin response sensor comprising:
at least two electrodes held in a fixed position on the skin of the subject;
at least one additional sensor; and
an impedance converter operatively connected to the at least two electrodes;
a microcontroller operatively connected to the impedance converter and to the at least one additional sensor wherein the microcontroller operates the impedance converter; and
a wireless transmitter operatively connected to the microcontroller;
obtaining at least one electrode measurement from the palm of the subject;
calculating at least one measured skin impedance for the subject using the at least one electrode measurement;
receiving a plurality of signals from the microcontroller encoding raw data comprising: the at least one electrode measurement; the at least one measured skin impedance, at least one additional sensor measurement from the skin impedance of the subject, and any combination thereof; and
transmitting a plurality of wireless signals encoding the raw data to the wireless receiver;
analyzing raw data using an analysis unit operatively connected to the portable unit, the analysis unit comprising:
a wireless receiver;
a computing device operatively connected to the wireless receiver, the computing device comprising:
at least one processor;
a computer readable media (CRM) encoded with a data analysis application executable on the at least one processor.

19. The method of claim 18, further comprising monitoring at least one additional state using the at least one additional sensor selected from the group consisting of a physiological state of the subject, a motion of the subject, and an ambient environmental condition.

20. The method of claim 18, wherein the CRM comprises a plurality of modules executable on the at least one processor, the plurality of modules comprising:
a signal processing module to smooth and/or filter the plurality of electrode measurements, the at least one measured skin impedance, and a second plurality of additional sensor measurements;
a data quality module to eliminate at least one artifact from the at least one measured skin impedance using a correction rule and second plurality of additional sensor measurements to produce at least one processed impedances;
a calibration module to convert the at least one processed impedances into at least one pain index value; and
a GUI module to generate one or more forms used to receive inputs to the analysis system and to deliver output from the analysis system.
